(19) United States
(12) Patent Application Publication
(10) Pub. No.: US 2005/0118287 A1
(43) Pub. Date: Jun. 2, 2005

(54) Use of a vanilloid as an anti-snoring element

(75) Inventors: Daniel Jean, Vic-Le-Vicomte (FR); Leon Cariel, Paris (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: LMD, VEYRE MONTON (FR)

(21) Appl. No.: 10/509,048

(22) PCT Filed: Mar. 25, 2003
(86) PCT No.: PCT/FR03/00936

(30) Foreign Application Priority Data
Mar. 25, 2002 (FR) 02/03681

(51) Int. Cl. A61K 35/78; A61K 31/36; A61K 31/12

(52) U.S. Cl. 424/756; 514/464; 514/685

(57) ABSTRACT

The invention relates to the use of a vallinoid to diminish, suppress or prevent snoring and in the production of a medicament for the treatment of sleep apnoea. Preferably, the vallinoid is selected from the group comprising capsaicin, piperine, gingerols and shogaols.

The present invention relates to novel compositions useful against snoring and sleep apnea. It relates in particular to the use of a vanilloid for preventing, reducing or suppressing snoring.

Except in the case of sleep apnea, snoring is not considered as a health problem. In general, it does not inconvenience the person who snores, but can greatly bother those around him. The snoring occurs more in men (20 to 32%) than in women (8%) (Jennum P. et al., J. Sleep Res., 1992, 1, p. 240-244 and Teculescu et al., Respiration 2001, 68(4), p. 365-370), and more in obese people than in slim people.

A typical snoring noise is produced when the soft tissues of the upper part of the respiratory tracts vibrate on the passage of air, which can be caused by one or more of four different situations:
the tissues (including the soft palate) which relax more than on average, and react as a flag or a sail which flaps in the wind;
excessive weight (in obese people) which causes sagging of throat, which hampers the passage of air;
polyps in the nasal tracts (a polyp is a longiform outgrowth which occurs on a mucous membrane);
obstructions, in the nasal tracts, caused by a cold or an allergy.

Some medicaments such as antihistamines or hypnotics can cause an excessive relaxation of the throat muscles and facilitate snoring. Sleeping on the back brings the tongue to the back of the palate and reduces the route for the passage of air, which can cause snoring.

Sleep apnea, for its part, consists of more or less long, more or less sustained respiratory arrests after which respiration resumes with noise. It is a problem which causes fatigue, mood swings, depression and even cardiac problems because of poor sleep and the dangerous reduction in blood pressure.

Currently, the problems of snoring are treated predominantly by surgery which is designed to modify the topology of the upper airways (veloplasty, somnoplasty, injection somnoplasty and the like). There are also various accessories which are thought to help snorers: pillows to better position the head, nasal orthesis for keeping the airways open, and the like. Some products for treating the problems of snoring are also provided, in particular essential oils, but without great success (WO 00/25588).

The applicant has discovered, surprisingly, that a vanilloid can be used to prevent, suppress or reduce snoring and to treat sleep apnea.

Shogaols which belong to the family of vanilloids, chemical substances related to vanillin, have already been described as having a deodorant and/or antiseptic activity (FR 2758086).

They are also known as aphrodisiacs (FR 01 02961).

Some shogaols are also known chemical constituents of plants of the genus Alpinia, such as Alpinia galanga or Alpinia officinarum or Zingiber, such as Zingiber officinalis, Zingiber cassumunar or Zingiber zerumbet, which are obtained by extraction of their rhizome.

Gingerol, another member of the vanilloid family, is also known as a constituent of ginger belonging to the Zingiber officinalis family. Anti-snoring compositions comprising, as main components, an extract of Zingiber officinale and Dioscorea (Dioscorea villosa or Dioscoreaceae inter alia) have also been described (U.S. Pat. No. 5,565,201, U.S. Pat. No. 5,603,935, U.S. Pat. No. 5,804,211).

However, the Zingiber officinale extract has never been described as having, on its own, an action against snoring. On the contrary, the presence of the second component appears to be essential (synergy of actions).

Furthermore, given the quantity of chemical constituents present in these extracts, nothing suggests that vanilloids alone can be anti-snoring active agents.

The present invention therefore relates to the use of a vanilloid to reduce, suppress or prevent snoring.

For the purposes of the present invention, the expression vanilloid is understood to mean any chemical molecule corresponding to either one of the following general formulae: 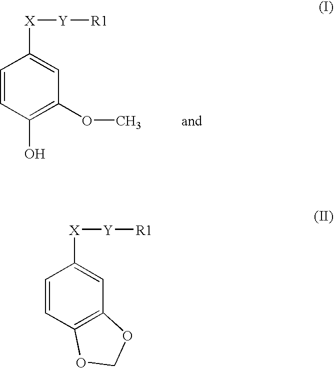
wherein
X represents a linear, saturated, monounsaturated or polyunsaturated, and substituted or unsubstituted C₁-C₄ alkyl group;
Y is chosen from the groups —N(H)C(O)—, —C(O)N—, and —C(O)—;
R1 represents a linear, branched or cyclic chain, saturated, monounsaturated or polyunsaturated, and substituted or unsubstituted C₂-C₂₄ alkyl group.

Advantageously, the vanilloid is chosen from the group consisting of capsaicin, piperine, gingerols and shogaols. Advantageously, this includes one or more shogaols.

Advantageously, the present invention relates to the use of a vanilloid for the manufacture of a medicament for treating sleep apnea.

Advantageously, the capsaicin corresponds to the following general formula:

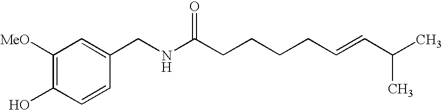

Advantageously, it is obtained by percolation of Capsicum fruit powder followed by purification of the capsaicin thus obtained. It is also commercially available.

Advantageously, the piperine is obtained by extraction of the pericarp of black pepper. It is also commercially available. Advantageously, the shogaol(s) correspond(s) to the general formula (III): 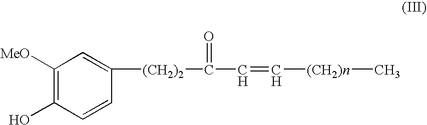
in which n is equal to 1, 2, 4, 6 or 8 and which are respectively called [3]-shogaol, [4]-shogaol, [6]-shogaol, [8]-shogaol and [10]-shogaol.

More advantageously still, this includes [3]-shogaol.

In a particular embodiment, the shogaol(s) are in the form of a crude extract of a plant of the Zingiberaceae family, advantageously by a process which comprises the following step:
a) preparation of the crude extract from fresh or dried rhizomes of said plant, by maceration of a ground product of these rhizomes at a temperature of between 10 and 35° C., followed by one or more extractions of this ground product under reflux, or by subjecting a ground product of said rhizomes to percolation at a temperature of between 10 and 35° C., each of these operations (maceration, extractions under reflux and percolation) being carried out by means of an organic solvent or of a mixture of appropriate organic solvents.

The maceration of the ground product of rhizomes, prior to its extraction, mainly has the effect of improving the bringing into contact of the plant tissues and of the solvent during the extraction. Its duration may be between about 12 hours and one week according to the state of freshness of the rhizomes used.

For the operations of maceration, extractions under reflux and percolation of the ground product, there are advantageously used organic solvents which are miscible with water and which have a relatively low boiling point so that they can be easily subsequently removed by mere evaporation, such as ethanol, methanol, acetone or mixtures thereof with water. However, since shogaols are soluble in numerous organic solvents, it is also possible to use other organic solvents such as ethyl acetate, ethyl ether, chloroform or methylene chloride.

In another particular embodiment, the shogaol(s) are in the form of a purified extract of a plant of the Zingiberaceae family, advantageously obtained by a process which comprises, in addition to step a) described above, the following additional steps:
b) purification of the crude extract obtained in step a) by subjecting said extract, after removal of the solvent(s) which it contains and its taking up in water, to one or more countercurrent extractions by means of organic solvent or of a mixture of organic solvents immiscible with water, and if desired,
c) chromatographic separation of the shogaols.

The water-immiscible organic solvent(s) used for carrying out the countercurrent extractions of the crude extract for the purpose of its purification are, for their part, chosen in particular from ethyl acetate, ethyl ether, chloroform, methylene chloride and mixtures thereof.

Advantageously, the plant of the Zingiberaceae family is chosen from the species Alpinia galanga, Alpinia officinarum, Zingiber officinalis, Zingiber cassumunar and Zingiber zerumbet, more advantageously still this includes Alpinia galanga.

For example, [3]-shogaol, [6]-shogaol and [8]-shogaol can be extracted from plants of the genus Alpinia such as Alpinia galanga or Alpinia officinarum, while [4]-shogaol and [10]-shogaol can be extracted from plants of the genus Zingiber such as Zingiber officinalis, Zingiber cassumunar or Zingiber zerumbet, using in particular the process as described above.

Advantageously, the crude extract of Alpinia galanga contains a quantity of [3]-shogaol by weight of between about 1 and 5% of the dry weight of said extract. In accordance with the invention, this extract is obtained from fresh or dried rhizomes of said plant.

Advantageously, the purified extract of Alpinia galanga contains a quantity of [3]-shogaol at least equal by weight to 75% of the dry weight of said extract.

The present invention also relates to the use of a vanilloid for the preparation of an anti-snoring composition.

Advantageously, the anti-snoring composition is formulated for oral administration, for example in the form of powders, solutions or suspensions to be taken orally, syrups, tablets or gelatin capsules.

Advantageously, the composition is formulated in the form of a nasal or mouth spray.

Physiologically acceptable excipients may be used in this type of composition. These excipients are conventional and well known to persons skilled in the art.

Advantageously, the vanilloid is used at a daily dose corresponding to 20 mg of vanilloid taken in the form of a plant extract or of pure vanilloid. Advantageously, shogaol is used in this case.

The following examples of preparation of extracts containing shogaols are given solely by way of illustrations of the subject of the invention and do not constitute in any manner a limitation thereto.

### EXAMPLE 1

### Preparation of a Crude Extract of Alpinia galanga Rhizomes

One kilo of fresh Alpinia galanga rhizomes is coarsely ground, being careful not to cause excessive heating of the ground parts. The water content of the ground product thus obtained is determined and it is subjected to maceration in 7 liters of ethanol whose strength is chosen such that, taking into account the water content of the ground product, the maceration solvent is 50% ethanol.

After 24 hours of maceration at about 20° C., the ground product is extracted under reflux with the maceration solvent for 30 minutes. This solvent is removed and replaced with an equal weight of 50% ethanol, and the ground product is again extracted under reflux for 30 minutes. The operation is repeated once.

The 3 extracts obtained are combined (thus constituting a volume of about 19 liters), filtered on paper and then evaporated to dryness under reduced pressure.

A residue is obtained which weighs about 50 g, that is an approximate yield of 30% relative to the dry weight of the rhizomes. This extract contains the various shogaols present in the Alpinia galanga rhizomes ([3]-shogaol, [6]-shogaol and [8]-shogaol) and its [3]-shogaol content is generally between 1 and 5% (w/w) depending on the rhizomes used.

### EXAMPLE 2

### Preparation of a Purified Extract of Alpinia galanga Rhizomes

50 g of a crude extract, prepared in accordance with example 1, are taken up in 1 liter of distilled water and the whole is brought to the boil for 1 minute with constant stirring. The stirring is continued until complete homogenization of this extract is obtained and it is left to cool. It is then subjected to 4 successive countercurrent extractions each carried out with 100 ml of ethyl ether.

The ethereal solutions are combined; they are supplemented with anhydrous sodium sulfate in order to remove the water which they contain; they are filtered on paper and evaporated to dryness under reduced pressure.

A residue is thus obtained which weighs 6.8 g, that is a yield of about 4% relative to the dry weight of the rhizomes. This extract, which contains predominantly [3]-shogaol has a [3]-shogaol content which is generally greater than 75% (w/w).

### EXAMPLE 3

### Production of [3]-shogaol

The [3]-shogaol can be obtained from Alpinia galanga rhizomes by preparing a crude extract of these rhizomes in accordance with example 1, by then purifying this extract in accordance with example 2 and then by subjecting the extract thus purified to successive elutions on silica gel columns, for example in the following manner.

10 g of a purified extract, prepared in accordance with example 2, are supplemented with 100 g of a G60 silica gel and 500 ml of chloroform with constant stirring. As soon as this mixture is homogeneous, it is evaporated to dryness under reduced pressure so as to obtain a powder.

This powder is deposited at the top of a column 10 cm in diameter and 50 cm in height, also containing G60 silica gel in petroleum ether. Elution is first carried out with petroleum ether until the residue is less than 0.1% (about 10 liters of petroleum ether necessary to reach this stage), and then with 12 liters of benzene and finally with 8 liters of chloroform.

The chloroform phase is evaporated to dryness under reduced pressure, leaving behind a residue of about 2.3 g. This residue is then subjected to preparative chromatography on a column 5 cm in diameter and 20 cm in height, filled with C18 silica gel, and using a water/acetonitrile (70/30) mixture as elution gradient. The fraction containing the [3]-shogaol is eluted within a period of between 5 and 7 minutes for a flow rate of 30 ml/min.

The [3]-shogaol can be identified by high-performance liquid chromatography (HPLC) coupled to mass spectrometry.

### EXAMPLE 4

### Preparation of an Anti-Snoring Composition in Oral Form

1 kilo of crude extract, prepared in accordance with example 1, was intimately mixed with 1 kilo of maltodextrin in a disk mill so as to give a better homogeneity to the mixture and to obtain a nonsticky free-flowing powder. This powder is then distributed into No. 0 gelatin capsules so as to obtain a unit dose of 250 mg of crude extract.

Likewise, the gelatin capsules may be manufactured from [3]-shogaol so as to obtain a unit dose of 25 mg of [3]-shogaol.

### EXAMPLE 5

### Activity of the Shogaol on Snoring

Daily doses corresponding to 20 mg of shogaol taken in the form of an extract according to example 1 or 2 or of pure shogaol according to example 3 were administered to a panel of men and women. The effects manifest themselves after 48 hours of treatment and persist for 48 hours after stopping the latter. Shogaol considerably reduces snoring in the user. Without being bound by theory, it appears that the effect of this molecule and of other vanilloids should be linked to their involvement in emission by certain nociceptive neurons of a neurotransmitter, substance P.

1-8. (canceled)

9. A method of reducing, suppressing, or preventing snoring comprising administering to a patient a vanilloid compound selected from the group consisting of the following formulae: 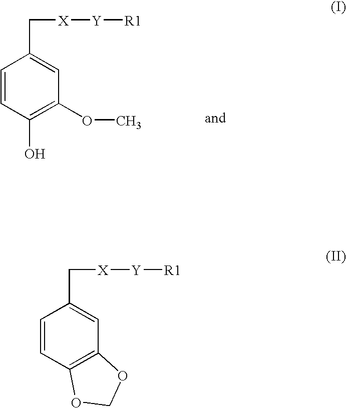
wherein
X represents a linear, saturated, monounsaturated or polyunsaturated, and substituted or unsubstituted C₁-C₄ alkyl group;
Y is selected from the group consisting of —C(O)N— and —C(O)—; and
R1 represents a linear, branched or cyclic chain, saturated, monounsaturated or polyunsaturated, and substituted or unsubstituted C₂-C₂₄ alkyl group.

10. The method of claim 9 wherein said vanilloid compound is selected from the group consisting of piperine, gingerols and shogaols.

11. The method of claim 10 wherein said vanilloid compound comprises at least one shogaol.

12. The method of claim 11 wherein said at least one shogaol corresponds to the general formula (III): 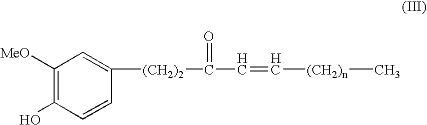
in which n is equal to 1, 2, 4, 6, or 8.

13. The method of claim 12 wherein n is 1.

14. The method of claim 11 wherein the at least one shogaol is in the form of a crude extract of a plant in the Zingiberaceae family.

15. The method of claim 14 including obtaining said crude extract by a process comprising the steps of:
(a) grinding the rhizomes of a plant in the Zingiberaceae family,
(b) macerating said ground rhizomes in an organic solvent or mixture of organic solvents at a temperature of between 10 and 35° C.; and
(c) either (i) extracting said ground rhizomes one or more times with an organic solvent or mixture of organic solvents under reflux, or (ii) percolating said ground rhizomes at a temperature of between 10 and 35° C. with an organic solvent or mixture of organic solvents.

16. The method of claim 11 wherein the at least one shogaol is in the form of a purified extract of a plant of the Zingiberaceae family.

17. The method of claim 16 including obtaining said purified extract by a process comprising the steps
(a) grinding the rhizomes of a plant in the Zingiberaceae family;
(b) macerating said ground rhizomes in an organic solvent or mixture of organic solvents at a temperature of between 10 and 35° C.;
(c) either (i) extracting said ground rhizomes one or more times with an organic solvent or mixture of organic solvents under reflux, or (ii) percolating said ground rhizomes at a temperature of between 10 and 35° C. with an organic solvent or mixture of organic solvents;
(d) removing said organic solvent or mixture of organic solvents;
(e) dissolving said extract in water;
(f) subjecting said extract in water to one or more countercurrent extractions by means of an organic solvent or mixture of organic solvents immiscible with water to provide a purified extract; and
(g) subjecting said purified extract to chromatographic separation to isolate the shogaols contained within said purified extract.

18. The method of claim 14 wherein said plant of the Zingiberaceae family is selected from the group consisting of the species Alpinia galanga, Alpinia officinarum, Zingiber officinalis, Zingiber cassumunar, and Zingiber zerumbet.

19. The method of claim 16 wherein said plant of the Zingiberaceae family is selected from the group consisting of the species Alpinia galanga, Alpinia officinarum, Zingiber officinalis, Zingiber cassumunar, and Zingiber zerumbet.

20. The method of claim 18 wherein said plant of the Zingiberaceae family comprises Alpinia galanga.

21. The method of claim 19 wherein said plant of the Zingiberaceae family comprises Alpinia galanga.

22. The method of claim 9 wherein said vanilloid compound is administered in the form of a nasal spray.

23. The method of claim 9 wherein said vanilloid compound is administered in the form of a mouth spray.

24. A pharmaceutical composition for the treatment of sleep apnea comprising a vanilloid compound selected from the group consisting of the following formulae: 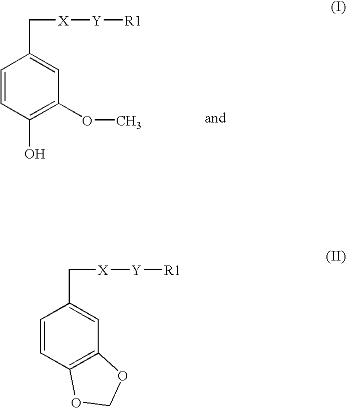
wherein
X represents a linear, saturated, monounsaturated or polyunsaturated, and substituted or unsubstituted C₁-C₄ alkyl group;
Y is selected from the group consisting of —C(O)N— and -zC(O)—; and
R1 represents a linear, branched or cyclic chain, saturated, monounsaturated or polyunsaturated, and substituted or unsubstituted C₂-C₂₄ alkyl group.
